(19) United States
(12) Patent Application Publication
(10) Pub. No.: US 2007/0092977 A1
(43) Pub. Date: Apr. 26, 2007

(54) FORENSIC TEST FOR HUMAN SALIVA

(76) Inventor: Karl Reich, Hillside, IL (US)

Correspondence Address: BAKER & MCKENZIE LLP, Pennzoil Place, South Tower, 711 Louisiana, Suite 3400, HOUSTON, TX, 77002-2716, US

(21) Appl. No.: 11/549,951

(22) Filed: Oct. 16, 2006

Related U.S. Application Data
(60) Provisional application No. 60/727,176, filed on Oct. 14, 2005.

(51) Int. Cl. G01N 33/558

(52) U.S. Cl. 436/514

(57) ABSTRACT

Lateral flow immunochromatographic strip tests for the detection of human saliva, method of detecting human saliva, and methods of manufacturing ICS tests for the detection of human saliva are described.

### PRIOR RELATED APPLICATION

This application claims priority to U.S. Provisional Application Ser. No. 60/727,176, filed Oct. 14, 2005, which is incorporated by reference herein in its entirety.

### FEDERALLY SPONSORED RESEARCH STATEMENT

Not applicable.

### REFERENCE TO MICROFICHE APPENDIX

Not applicable.

### FIELD OF THE INVENTION

The invention relates to lateral flow test strips that are both sensitive and accurate for the detection of human saliva. Lateral flow test strips provide a rapid method for confirmation of human saliva in forensic samples.

### BACKGROUND OF THE INVENTION

The total volume of saliva produced each day in adults is 500 to 1500 ml. Almost all of the organic compounds of plasma, such as hormones, immunoglobulins, enzymes, DNA and viruses may be detected in saliva in trace amounts. Saliva is also an adequate source of DNA for analysis such as DNA typing in certain forensic settings. An assay for human saliva in a forensic sample is required to document qualitatively the presence of human saliva and determine if further analysis is warranted.

Recently, enzyme linked immuno-absorbant (ELISA) assays have been developed to detect human salivary amylase (Quarino, et al., 2005) and electronic biosensors are also being developed (Aluoch, et al. 2005). These assays require a clinical setting, complex materials, or increased sample volume, and are not easily interpreted.

A sensitive, specific, quantitative, and rapid assay of human saliva is required for forensic applications.

### SUMMARY OF THE INVENTION

This invention relates generally to the detection of human saliva, and is used in criminal and civil forensics for the definitive detection of human saliva. It uses a lateral flow test that employs antibodies for human salivary amylase (hAMY1A), an enzyme found primarily in human saliva. The test can use either monoclonal antibodies, recombinant antibodies, or polyclonal antibodies configured into capture and detection components. It is the first such test available for species specific identification of human saliva and is generally useful for the identification of saliva from a wide variety of samples.

In one embodiment, the invention involves the use of two monoclonal antibodies that each recognize a single non-overlapping epitope unique to hAMY1A. The antibodies are configured in a lateral flow immunochromatographic strip (ICS) test that is generally described as follows: The capture antibody is immobilized on the test strip at a defined test line position. The detection antibody is labeled for visualization, and is placed on the test strip at the origin. Samples to be tested for the presence of human saliva are deposited onto the test strip at the origin, and a complex of hAMY1A and labeled detection antibody is then formed. The complex migrates along the test strip by capillary action and is captured by the immobilized capture antibody as it passes thereby. The complex, initially diffused at the origin of the test strip, becomes visible to the naked eye as it is concentrated at the test line. The test is rapid, sensitive and specific for hAMY1A and provides a definitive test for the presence of human salivary amylase.

Human salivary amylase described at GenBank Acc # NP_{—}004029, incorporated by reference, may also be referred to as alpha-1 A salivary amylase, 1,4-alpha-D-glucan glucanohydrolase, glycogenase, and alpha-amylase.

In one embodiment, human salivary ICS tests can be manufactured by placing a backing on one surface and laminating a conjugate pad with conjugated detection antibodies that bind human salivary amylase (hAMY1A) at one end of the backing, a wick (or absorbent pad) opposite the conjugate pad, and a chromatographic membrane with a test line and a control line connecting the wick and conjugate pad. The test line has immobilization antibodies that bind hAMY1A and the control line has anti-Ig antibodies that bind detection antibody/conjugate complex or free antibody.

Detection antibodies can be polyclonal antibodies, monoclonal antibodies, recombinant antibodies, AMY1, AMY2, or AMY16C. Detection antibodies are labeled for detection by a variety of known methods. Generally they are conjugated with colloidal gold, streptavidin, biotin, microspheres, peroxidase, horse radish peroxidase (HRP), streptavidin-labeled HRP, phosphatase, alkaline phosphatase (AP), chromogenic labels, fluorescent labels, chemiluminescent labels, phosphorescent labels and the like.

Immobilization antibodies may also be polyclonal antibodies, monoclonal antibodies, recombinant antibodies, AMY1, AMY2, or AMY16C.

A sample pad may optionally be placed adjacent to or atop the conjugate pad to remove solid debris from the tested sample.

A sample test strip can be used to verify control concentrations of hAMY1A from 0 ng/ml to 100 ng/ml, the negative control being 0 ng/ml hAMY1A.

The ICS tests can be assembled in a cassette with one or more strips in a cassette for parallel tests. This is particularly useful for a cassette containing a negative (−) control without antigen and/or a positive (+) control with a known concentration of hAMY1A. ICS tests may also be provided in a kit with instructions, sample swabs, swab wetting solution, sample tubes, sample tube holder, scissors, sample extraction buffer, sample running buffer, transfer pipet, or completed ICS test documentation envelope, and the like.

### BRIEF DESCRIPTION OF THE DRAWINGS

FIG. 1: Lateral Flow Immunochromatographic Strip Test for Detection of Human Saliva.

### DETAILED DESCRIPTION OF THE INVENTION

The present invention provides an immunochromatographic strip (ICS) test for the presence of hAMY1A, a method to determine the presence of human saliva using an ICS test and a method of manufacturing an ICS test for hAMY1A. The ICS test consists of two antibodies specific for hAMY1A, one of which functions as a detection antibody and the other an immobilization or capture antibody.

In its simplest form, the ICS is an absorbent pad containing diffuse detection antibodies and one location with fixed immobilization antibodies (a test spot or line). Antigen in a sample is carried by capillary action to the immobilization antibodies. The once diffuse detection antibodies are complexed with the antigen and become concentrated at the site of fixed immobilization antibodies. The presence of antigen is confirmed as detection antibodies become visible or are otherwise detected.

To improve the intensity of the detection signal, different media can be used. In one embodiment, a conjugation pad is used to retain the conjugated detection antibodies until sample is added. Nitrocellulose, PVDF, or other membrane may be used to bind the immobilization antibodies in a fixed location. To encourage capillary action, a wick may be placed opposite the conjugate pad or sample location.

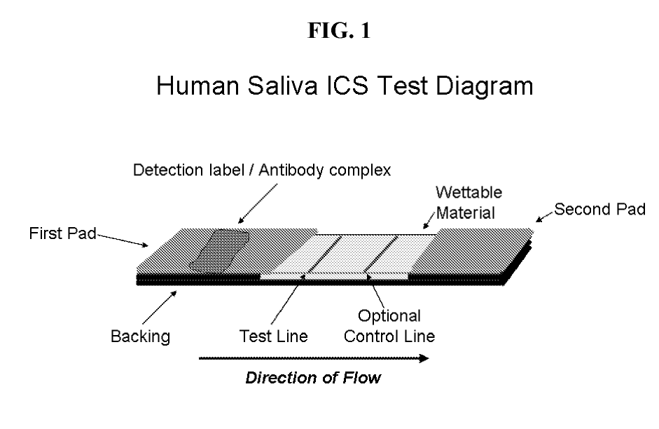

FIG. 1 depicts the design of a lateral flow test using specific hAMY1A antibodies. As shown in FIG. 1, the three components of the lateral flow test are the conjugate pad, the membrane, and the wick. The conjugate pad can be made from absorbent material such as glass-fiber paper, rayon, cotton or polyester. The membrane can be made from nitrocellulose, PVDF, or other material with or without a non-absorbent backing. The wick can be made from absorbent material such as cellulose, glass-fiber paper, rayon, cotton or polyester. A sample pad is sometimes used in addition to the conjugate pad and functions as a primary filter for complex extracts. The sample pad is placed above the conjugate pad and can be made from absorbent material such as glass-fiber paper, rayon, cotton or polyester. The materials required for conjugate pads, membranes, wicks, and sample pads are available commercially from WHATMAN® , MILLIPORE® , SIGMA® , or other sources and may be optimized for specific antibody combinations.

In one embodiment, the conjugate pad contains detection antibodies labeled with colloidal gold, latex beads, fluorescent compounds, enzymes, biotin or other reagents commonly used for immunological visualization or visual enhancement. The membrane is configured to contain a ‘control line’ and a ‘test line’ . The test line contains immobilized hAMY1A antibody that binds the labeled hAMY1A conjugate. The control line contains an anti-Ig or other appropriate control antibody (i.e. anti-mouse, anti-rabbit, anti-goat, anti-sheep or anti-horse) and detects unbound detection antibody and antibody conjugate complexes, thus confirming contents of the conjugate pad including test sample have immunochromatographed past the test line. The wick soaks up excess sample to prevent backflow during the detection time window.

Antibodies are specific for hAMY1A and may be generated from numerous sources using techniques known to one of ordinary skill in the art. Preferably, the antibodies do not cross-react with the saliva of ferret, domestic cat, rabbit, cow, dog, goat, skunk or opposum (Mustela nigripes, Felis catus, Oryctolagus cuniculus, Bos taurus, Canis familiaris, Capra hircus, Mephitis mephitis, Didelphis virginiana, respectively) and do not cross react with human pancreatic amylase. Antibodies are commonly generated in mice, rats, rabbits, goats, or horses, but can come from a variety of animals. Further, it is preferred that the two antibodies bind non-overlapping epitopes and can simultaneously bind to hAMY1 A.

Antibodies may be polyclonal, monoclonal or recombinant antibodies. Polyclonal antibodies are the most readily available and least expensive to produce, but do not bind to a specific epitope. Multiple binding sites provide less specificity and multiple molecular targets, but provide detection of smaller samples as multiple antibodies bind one protein molecule. Monoclonal antibodies bind a single epitope, are more specific and are more expensive to produce. Recombinant antibodies are the most difficult to produce as the light and heavy chains must be cloned into a single recombinant coding sequence. These antibodies may be engineered to increase sensitivity or provide a commercially detectable marker (His-tag or other protein marker).

In one embodiment, both ‘capture’ and ‘detection’ antibodies are identified by simultaneous binding with authentic hAMY1A. In another embodiment, the invention is directed to monoclonal antibodies AMY1, or AMY2 as well as polyclonal antibody AMY16C, as either the capture or detection antibody. Alpha-amylase antibodies with varying specificity for human alpha-amylase are commercially available from ABCAM® (www.abcam.com), ROCHE® , and other commercial sources.

A method of detecting human saliva in a test sample suspected of containing human saliva is described. Samples may be taken from various surfaces where human saliva may be present including cans, glasses, cups, cigarette butts, lipstick, plastic or metal, and solid or porous surfaces. Samples may be wet, moist, or dried. Dried or moist samples can be collected with a wet swab. Wet samples may be assayed directly or swabbed. Aqueous samples are extracted from swabs by adding buffer. Aqueous samples may be stored in a sealed container at 4° C. or frozen for long term storage. Lyophilized samples can be resuspended and assayed at a later date. A volume of sample is applied to the conjugate pad, the sample is allowed to migrate across the membrane, and the presence of hAMY1A is determined. In one embodiment a sample containing a known amount of hAMY1A is compared to the test sample (positive control), thus confirming activity of the test.

In one embodiment, an ICS is manufactured on a backing with a first pad (conjugate), a second pad (wick), and a membrane between the conjugate pad and wick (FIG. 1). The wick is applied to one end, the membrane is in physical contact with the wick, and the membrane extends to the opposite end of the strip. The conjugate pad is placed in physical contact with the membrane opposite the wick. Electro-mechanical dispensers are used to dispense the antibody complexes onto the conjugate pad, as well as apply the test and control lines on the membrane. Antibodies may be applied to the conjugate pad and membrane prior to assembly on solid support or after the wick, membrane, and conjugate pad are partially assembled. In one embodiment, membranes are pre-treated with control and test lines, blocked with ovalbumin, BSA or milk proteins, and optionally crosslinked to covalently bind capture antibody to the membrane. Optional sample pad or cassette case may be added to complete manufacture. One or more ICS tests may be combined with instructions, sample swabs, swab wetting solution, sample tubes, sample tube holder, scissors, sample extraction buffer, sample running buffer, transfer pipet, completed ICS test documentation envelope, syringes, immunochromatographic solutions, additional detection antibodies, secondary enzyme assay reagents, and the like.

### EXAMPLE 1

Prepartion of Polyclona Antibodies

Polyclonal antibodies were produced in New Zealand white rabbits by repeated intradermal injections of hAMY1A-Freud' adjuvant mixture (complete Freud's adjuvant for the first injections series and incomplete Freud's adjuvant for subsequent injections series). Human salivary amylase (a-Amylase # A1031, Enzyme Commission (EC) Number 3. 2. 1. 1) was prepared as an emulsion with Freud's adjuvant (complete or incomplete) and repeatedly passed between two glass syringes using a straight pass-through uer-lock syringe connector. Emulsions were ready for injection when the effort required to pass the solution across the syringe bridge is almost beyond the operator's ability. Injections were performed with 22 or 23 gauge needles intradermally at multiple locations on the rabbit's back. Booster injections with antigen in Freud's incomplete adjuvant were performed at standard intervals (2-3 weeks). Animals were bled, usually through the marginal ear vein, and the serum collected after overnight storage. Serum was analyzed by ELISA and developed with HRP conjugated goat-anti-rabbit antibodies. ELISA positive rabbits received additional antigen boosts and were bled at regular intervals alternating with booster injections. Antibodies were purified from selected rabbits by precipitation with ammonium sulfate and purified further by benzamidine agarose chromatography.

### EXAMPLE 2

Prepartion of Momoclonal Antibodies

BALB/C mice were immunized with a hAMY1prepared in Freud's adjuvant as previously described. A two to three week schedule of immunization and boosters was used. Responder animals were identified by tail vein bleeds and analysis by ELISA. Positive responder animals received one additional boost and were prepared for splenic fusion.

Spleen cells were isolated from sacrificed animals and fused with previously prepared myeloma cells. Cell fusion was carried out by co-centrifugation of freshly harvested spleen cells with myeloma cells in polyethylene glycol, and subsequent distribution into 96 well plates with or without feeder layers.

Culture supernatants from surviving fusions, i.e., individual wells, were tested for mouse IgG production and for production of IgG that recognizes hAMY1A by ELISA or similar immunological assay. Myeloma fusion cells from Ig producing antigen positive wells were cloned by limiting dilution, grown and retested for Ig production and for specificity to the immunization hAMY1A before being expanded. Cloning by limiting dilution can be repeated to ensure that antibody producing cells are true clones. Generally four to six cell cultures of each Ig positive clone were prepared and stored in liquid nitrogen. Antibodies from selected wells were tested in various combinations to identify clones that produce antibodies to non-overlapping epitopes.

### EXAMPLE 3

Collodial Gold Labeling

Colloidal gold solution was monitored by optical absorbance at 521-525 nm. For 2L of colloidal gold solution 24 ml of 1.14% gold chloride and 40 ml of 1.14% sodium citrate were mixed in a baffled 4L flask with 18.2 mega ohms pure water (MILLIPORE® filtered).

1. Boil 2.28 L pure water; trap water vapor to prevent loss of volume;

2. Start timer: at 30 seconds add 24 ml 1.14% gold chloride; increased stirring;

3. At exactly 2 minutes, add 40 ml 1.14% sodium citrate; and

4. Continue stirring for 3 minutes until cherry red, for a total of 5 minutes.

The solution went through various color changes. Upon addition of the gold chloride, the solution was yellowish; upon addition of the sodium citrate, the solution went from yellow to clear (at about 2 min 30 sec). At about 3 minutes, solution was completely black, then purplish, then at about 4 minutes it appeared reddish-purple. At 5 minutes, the solution was a deep cherry red. When the solution was cherry red, the flask was cooled in a water bath. For gold chloride solution a Teflon coated spatula was used because Au₃Cl will corrode metal spatulas.

Optical density of gold colloidal solution was scanned from 400-600 nm and the peak and wavelength maxima noted. Typical optical density (OD) values of peak absorbance were about 1.1 OD at 525 nm.

Conjugation of colloidal gold particles to antibodies was empirically determined for each batch of antibodies and was sensitive to the pH and pI of the antibodies. Trial solutions of 7.5 ml colloidal gold were prepared at pH of 7.0, 7.8, 8.2, 8.6, and 9.0. Potassium carbonate (0.2 M) was used to adjust pH of colloidal gold solution from about pH 4.0 and additional colloidal gold solution was used to adjust the solution pH if required, because other acids cause precipitation of the colloidal gold. Antibody solutions were prepared at 0.2 mg/ml and diluted with 2 mM phosphate buffer, pH 9.0. Test solutions with no antibody (0) as well as 6 μg/ml, 8 μg/ml, 10 μg/ml, and 12 μg/ml solutions of antibody were analyzed with the above pH ranges of colloidal gold (Table 1).

TABLE 1
COLLOIDAL GOLD LABELING

| | | | | |
|---|---|---|---|---|
| Antibody | Ab (ml) | Buffer (ml) | Gold | NaCl |

| | | | | | | | |
|---|---|---|---|---|---|---|---|
| 0 | | 0 | | 50 | ml | 500 μl | 100 μl |
| 6 | μg/ml | 16.5 | ml | 33.5 | ml | 500 μl | 100 μl |
| 8 | μg/ml | 22 | ml | 28 | ml | 500 μl | 100 μl |
| 10 | μg/ml | 27.5 | ml | 22.5 | ml | 500 μl | 100 μl |
| 12 | μg/ml | 33 | ml | 17 | ml | 500 μl | 100 μl |
| 16 | μg/ml | 44 | ml | 6 | ml | 500 μl | 100 μl |

Antibody and colloidal gold were mixed and allowed to stand at room temperature for 2 minutes. Sodium chloride (10% w/v in H₂O, pre-filtered 0.22 μm) was added and allowed to stand for 5 minutes. The optical density (OD) of each solution was measured and λ max recorded. The optimal ratio of antibody/colloidal gold was determined by identifying the lowest concentration of antibody that did not precipitate unbound colloidal gold particles on the addition of salt.

### EXAMPLE 4

Conjugation

Anti-hAMY1A antibodies “AMY1” (Tu66C7, Cat# 11543 601) and “AMY2” (Tu88E8, Cat# 11543 598) were purchased from ROCHE DIAGNOSTICS® GmbH (Mannheim, Germany).

200 micrograms of AMY1 antibody at a concentration of 10 μg/ml was mixed with 20 ml colloidal gold, pH 8.2, and gently rocked for 30 minutes. After 30 min, 2.2 ml of 10% BSA (for 1% BSA final concentration) was added and gently rocked for 1 hour.

320 micrograms AMY2 antibody at a concentration of 16 μg/ml concentration was added to 20 ml colloidal gold, pH 9.0, and gently rocked for 30 minutes. After 30 min, 2.2 ml 10% BSA (for 1% BSA final concentration) was added and gently rocked for 1 hour.

### EXAMPLE 5

Treatment of Conjugate Pad

Glass fiber conjugate pad material was cut into strips 300 mm long by 22 mm wide. To make the conjugate pad more hydrophilic, the strips were pretreated by immersion in a solution containing sodium tetraborate (0.2%), Bovine Serum Albumin (BSA, 3%), polyvinylpyrrolidone (PVP, 1%), sucrose (0.1%), and Triton X-100 (0.25%) for 10 minutes (5 minutes on each side). The treated pads were blotted dry and dried in an oven at 37° C. for one hour. The dried conjugate pads were stored in an airtight and moisture resistant foil pouch containing desiccant.

### EXAMPLE 6

Lateral Flow Strip Test Materials

A variety of test materials were used to identify the optimal combination of membrane, conjugate pad and blocking solutions for each antibody combination. These include different nitrocellulose membranes, different conjugate pad materials, different mixtures of detergents and buffers for blocking. The use of sample pads must also be tested depending on the sample origin.

### EXAMPLE 7

Assembly of Lateral Flow Strip Test

The components of the test strip were assembled on cards, then cut into strips of a desired width. A lamination instrument with a lid and a base that use vacuum pressure to hold components in the correct orientation was used to assemble cards. The lid holds a backing card with adhesive facing outward and the base holds the components of the strip test. Components were placed in pre-machined grooves on the base in reverse order determined by the geometry of the laminator. In one example, the components were assembled as follows:

1. The sample pad in the top groove near the hinge;

2. The conjugate pad in the next slot, overlapping the sample pad;

3. The wick in the opposite slot; and

4. The membrane in the middle slot, with the edges of the membrane overlapping the wick and the conjugate pad by ˜2mm.

The top of the laminator was pressed down to adhere the components together. Assembled and laminated cards were cut into 3-5 mm sections using a dedicated step motor driven guillotine cutter. Assembled cards were fed into the device between the material guide rails and advanced until the front edge of the assembled card reaches the closed blade. Cards were fed automatically through the cutter to generate strips of a preset width. Cut strips were stored over desiccant until use or assembled into cassette cases. One or more test strips may be assembled into a cassette strip for parallel assays.

### EXAMPLE 8

Concentration of Colloidal Gold Conjugate

AMY1 and AMY2 gold conjugate was spun down at 16,000 G for 25 minutes with no brake at 4° C. Supernatant was carefully removed and 1.8 ml of passive gold diluent (0.07% sodium phosphate and 0.1% BSA) was added for an approximate 10-fold dilution. The OD of the colloidal gold conjugate was adjusted to about 10 absorbance units.

### EXAMPLE 9

Conjugate Pad Preparation

20% w/v sucrose and 5% w/v trehalose was added to 1 ml AMY1- and AMY2-conjugate. AMY1 and AMY2 conjugate were dispensed onto a pretreated conjugate pad at 10 μl/cm using an airjet Quanti Dispenser. After dispensing the conjugates onto the pads, the conjugate pads were dried at 37° C. for 1 hour.

### EXAMPLE 10

Ics Membranes Preparation

AMY1 and AMY2 were dispensed onto a nitrocellulose membrane using a BIODOT QUANTI® dispenser at 1 μl/cm and lmg/ml antibody solution. Goat anti-mouse was dispensed onto the same membrane as either AMY1 or AMY2, 4 mm above the test line (called the control line). The membranes were dried at 37° C. for 1 hour. The membranes were blocked by immersion in a solution of 0.25% PVP, 0.1%BSA, and 0.1% sucrose in 10 mM phosphate buffer. After blocking, the membranes were dried at 37° C. for 1 hour.

### EXAMPLE 11

Sandwich Elisa

To prove the antibodies bind distinct epitopes, sandwich ELISA experiments were conducted. Antibodies were assayed in the capture and detection orientation. The ELISA assay tested salivary amylase cognate antigen, human saliva extracts from buccal swabs, and human blood to show saliva specificity (Table 2).

Human saliva was collected by swabbing the inside of a human cheek and allowed to air dry overnight. Human blood (50 μl) was placed on a cotton swab and allowed to air dry overnight. Each of the swabs, buccal and blood, was extracted in ˜250-1000 μl of phosphate buffered saline (PBS) for a minimum of 1 hour. Magnetic beads were labeled with capture antibody and diluted into blocking buffer (1% ovalbumin) for 1 hour. The blocked, capture antibody-labeled beads were placed in each well of a 96-well plate. A sample of hAMY1A antigen was added and brought to a final volume of ˜ 100 μ. After a 2 hour incubation at room temperature, the beads were washed four times in TBST (tris-buffered saline with triton). The biotinylated detection antibody was added to each sample and allowed to incubate for 1 hour at room temperature. The beads were washed four times with TBST. Streptavidin-labeled Horse Radish Peroxidase (HRP) was added to each sample and allowed to incubate at room temperature for 30 minutes. The beads were washed four times with TBST. Ortho-Phenylenediamine (OPD) was added to the washed beads and allowed to incubate for 10 minutes at room temperature. The calorimetric reaction and absorbance change was monitored at 490 nm. A signal to noise calculation was then determined by normalizing the sample well absorbance to a control well with no antigen for background absorbance.

Regardless of capture/detection orientation, AMY1 and AMY2 were able to detect human saliva ˜5-12 times above background levels and showed no cross-reactivity with human blood. This demonstrates AMY1 and AMY2 have two distinct epitopes on the monoclonal salivary amylase antibodies and low cross-reactivity with human blood.

TABLE 2
ELISA SANDWICH ASSAY

| | AMY1 Capture/ | AMY 2 Capture/ |
|---|---|---|
| | AMY 2 Detection | AMY1 Detection |
| Sample | (Signal to Noise) | (Signal to Noise) |
| No Antigen Control | 1.00 | 1.00 |
| hAMY1A Cognate | 3.55 | 8.19 |
| Antigen (5 ng) | | |
| Human Saliva (1 μl) | 4.89 | 12.17 |
| Human Saliva (5 μl) | 5.03 | 12.23 |
| Human Blood (1 μl) | 1.16 | 1.00 |
| Human Blood (5 μl) | 1.43 | 1.62 |

### EXAMPLE 12

Antibody, Membrane and Pad Optimization

Antibody detection systems for human saliva were optimized for antibody orientation, membrane composition, and conjugate pad conditions (Table 3). For antibody orientation, combinations of available capture and detection antibodies were assayed. Antibodies AMY1 and AMY2 were assayed to determine the best configuration for capture and detection of human amylase. For membrane and conjugate pad selection, multiple candidates for both product materials were tested and scored. For buffer selection, various salt concentrations, pH ranges, and blocking agents were tested to determine the recipe that gave minimal background and maximum sample signal with human saliva.

TABLE 3
OPTIMIZATION

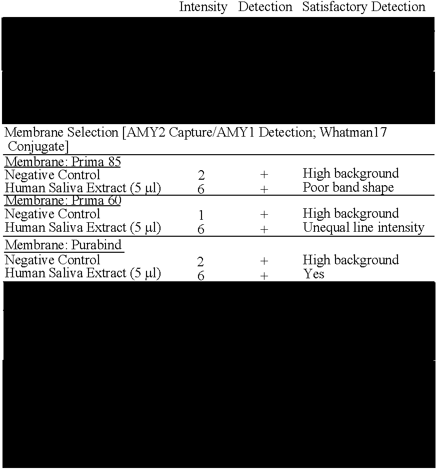

### EXAMPLE 13

Saliva Detection Capability

These experiments address human saliva detection from various exhibits and are designed to mimic the forensic field application of ICS tests. To ensure the ability of the lateral flow ICS tests to detect human saliva, extracts from multiple exhibits were tested including buccal swab, plastic bottle, plastic mug, ceramic mug, cigarette butt, and soda can (Table 4). All sample exhibits that contained saliva resulted in positive detection results, demonstrating broad applicability of hAMY1A ICS tests for human saliva detection on a variety of surfaces.

TABLE 4
SALIVA DETECTION CAPABILITY

| | | | Satisfactory |
|---|---|---|---|
| Sample | Intensity | Detection | Detection |
| Human Saliva (Swab) | 5 | + | Yes |
| Human Saliva (Plastic Bottle) | 4 | + | Yes |
| Human Saliva (Plastic Mug) | 5 | + | Yes |
| Human Saliva (Ceramic Mug) | 6 | + | Yes |
| Human Saliva (Cigarette Butt) | 8 | + | Yes |
| Human Saliva (Soda Can) | 5 | + | Yes |

### EXAMPLE 14

Body Fluid Specificity Experiment

Cross-reactivity of hAMY1A ICS tests with human body fluids from blood, semen, urine, breast milk, sweat, feces, ear wax and amniotic fluid were assessed to ensure the specificity for human saliva (Table 5). No cross-reactivity was observed with the body fluids tested, other than breast milk and feces which are known to contain small amounts of amylase (Muller G. Z. Gastroenterol. 1980. 18:198-202, Heitlinger et al., Pediatric Research. 1983. 17:15-18.)

TABLE 5
BODY FLUID SPECIFICITY

| Sample | Intensity | Detection | Satisfactory Detection |
|---|---|---|---|
| Human Saliva | 5 | + | Yes |
| Human Blood | 0 | − | Yes |
| Human Semen | 0 | − | Yes |
| Human Urine | 0 | − | Yes |
| Human Breast Milk | 3 | + | Yes |
| Human Sweat | 0 | − | Yes |
| Human Feces | 2 | + | Yes |
| Human Ear Wax | 0 | − | Yes |
| Human Amniotic Fluid | 0 | − | Yes |

### EXAMPLE 15

Breast Milk Cross-Reactivity Experiment

To more rigorously investigate the reported low-level cross reaction between hAMY1 A immunigraphic test strips and breast milk, reactions with breast milk were directly compared to saliva (Table 6). Breast milk was collected in a manner that was designed to minimize the chance of saliva contamination by washing the skin area and collection of breast milk without the use of a breast pump. Samples were collected from a participating laboratory under informed consent. Although breast milk demonstrated a consistent low reaction with hAMY1A immunographic test strips, breast milk was approximately 20 fold less reactive that human saliva.

TABLE 6
BREAST MILK CROSS-REACTIVITY

| | | |
|---|---|---|
| Strip | Extract Analyzed | Results |

| | | | |
|---|---|---|---|
| 1 | 20 | μl sham | 0/10 |
| 2 | 20 | μl saliva | 9/10 |
| 3 | 10 | μl saliva | 8/10 |
| 4 | 5 | μl saliva | 7/10 |
| 5 | 1 | μl saliva | 4/10 |
| 6 | 20 | μl breast milk | 2/10 |
| 7 | 10 | μl breast milk | 2/10 |
| 8 | 5 | μl breast milk | <1/10 |
| 9 | 1 | μl breast milk | <1/10 |

### EXAMPLE 16

Species Specificity Experiment

Cross-reactivitiy of hAMY1 A immunographic test strips with non-human saliva wass assessed with saliva from a variety of species to ensure the specificity of the test strip (Table 7). Human saliva detection was compared to a variety of species including tamarin, opossum, ferret, domestic dog, callimico, horse, chameleon, llama, goat, sheep, marsh snake, hedgehog, domestic pig, domestic rabbit, mongoose, and grey gull. No cross-reactivity was observed with a variety of animal saliva samples tested.

TABLE 7
SPECIES SPECIFICITY

| Sample | Intensity | Detection | Satisfactory Detection |
|---|---|---|---|
| Human Saliva | 5 | + | Yes |
| Tamarin Saliva | 0 | − | Yes |
| Opossum Saliva | 0 | − | Yes |
| Ferret Saliva | 0 | − | Yes |
| Domestic Dog Saliva | 0 | − | Yes |
| Callimico Saliva | 0 | − | Yes |
| Horse Saliva | 0 | − | Yes |
| Chameleon Saliva | 0 | − | Yes |
| Llama Saliva | 0 | − | Yes |
| Goat Saliva | 0 | − | Yes |
| Sheep Saliva | 0 | − | Yes |
| Marsh Snake Saliva | 0 | − | Yes |
| Hedgehog Saliva | 0 | − | Yes |
| Domestic Pig Saliva | 0 | − | Yes |
| Domestic Rabbit Saliva | 0 | − | Yes |
| Mongoose Saliva | 0 | − | Yes |
| Grey Gull Saliva | 0 | − | Yes |

### EXAMPLE 17

Hook Effect

Limit detection capabilities are often observed as the high dose Hook effect. The mechanism of high dose Hook effect is not well understood, but as the concentration of sample is increased an upper limit of detection is often observed with ICS tests. Often samples containing overwhelming amounts of antigen are not detected, giving false negative results. To assess the high dose Hook effect of the ICS test for human saliva, sample swabs were prepared by placing 50 μgl of human saliva on a swab and air drying overnight. Sample swabs were immersed in 200, 400, or 1000 μl of extraction buffer and incubated at room temperature for a minimum of 1 hour. Samples were then assayed on ICS tests and scored as described for test line intensity (Table 8). In the relevant range for forensic detection of human saliva, the ICS test shows no significant high dose Hook effect. This lack of high dose Hook effect ensures no false negative results when testing high concentrations of human saliva.

TABLE 8
HOOK EFFECT

| | Extract | | Volume | | Equivalent | | Inten- | Detection |
|---|---|---|---|---|---|---|---|---|
| | Volume | | Applied | | Saliva | | sity | Result |
| Negative Control | 1 | ml | 25 | μl | 0 | μl | 0/10 | − |
| Human Saliva | 1 | ml | 5 | μl | 0.25 | μl | 8/10 | + |
| Extract | | | | | | | | |
| Human Saliva | 1 | ml | 25 | μl | 1.25 | μl | 9/10 | + |
| Extract | | | | | | | | |
| Human Saliva | 1 | ml | 50 | μl | 2.5 | μl | 9/10 | + |
| Extract | | | | | | | | |
| Human Saliva | 1 | ml | 75 | μl | 3.75 | μl | 9/10 | + |
| Extract | | | | | | | | |
| Human Saliva | 1 | ml | 100 | μl | 5 | μl | 9/10 | + |
| Extract | | | | | | | | |
| Human Saliva | 400 | μl | 50 | μl | 6.25 | μl | 9/10 | + |
| Extract | | | | | | | | |
| Human Saliva | 400 | μl | 100 | μl | 12.5 | μl | 8/10 | + |
| Extract | | | | | | | | |
| Human Saliva | 200 | μl | 100 | μl | 25 | μl | 8/10 | + |
| Extract | | | | | | | | |

In the above assays, each of the hAMY1A ICS tests was subjected to visual analysis to ensure control line uniformity and intensity, test line uniformity and intensity, intensity equality between control line and test line, long term strip stability and other factors that affect the appearance of the test line and control line. Tests were then analyzed for Test Line Intensity, Detection Results, and Satisfactory Detection. Test Line Intensity for each test was scored by an internally established intensity scale of 1-10 at the end of the 10 minutes. Detection Results were assigned a positive (+) or negative (−) signal detection based on test line intensity score. A test line intensity score of “0” is assigned a negative result (−), while a test line intensity score of “1-10” is assigned a positive result (+). Satisfactory Performance was assayed based on commercial applicability under the various conditions.

For control assays, single strips were placed in glass tubes containing 100 μl of a specified test solution with the conjugate side to the bottom of the glass tube and the liquid diffused up the strip. The reaction was stopped by removing the strips from the glass tubes, laying the strips on a paper towel, and removing the conjugate pad with forceps. The intensity of the test and control lines were quantified using an internally established intensity scale of “0-10” after the 10 minute detection time window, “0” being signal from negative (−) control and “1-10” being signal from various concentrations of positive (+) control.

All sample swabs were prepared by placing 50 μl of tested substance on the swab head and allowed to air dry overnight. Substance exhibits were prepared by swabbing stained material and allowing the swab to air dry overnight. All sample swabs were then immersed in 250-1000 μl of extraction buffer and incubated at room temperature for a minimum of 1 hour.

### EXAMPLE 18

Saliva Detection Kit

Human salivary amylase immunographic test strips may be supplied with all reagents in a simple-to-use kit designed for laboratory or field use. The ability to rapidly detect saliva presence without requiring a laboratory setting provides a powerful tool for forensic applications. Saliva can be located on site and further analysis conducted. One or more hAMY1A ICS will be supplied with some or all of the following components: sample swabs, swab wetting solution, sample tubes, sample tube holder, scissors, sample extraction buffer, sample running buffer, transfer pipet, completed ICS test documentation envelope, syringes, immunochromatographic solutions, additional detection antibodies, or secondary enzyme assay reagents, and the like. Instructions may also be included. One or more ICS tests in cassettes may be provided in the same cassette.

In one embodiment, an area suspected of containing saliva would be identified. A moistened sample swab is applied to the area and transferred to a sample tube. Sample buffer can be added to extract the sample. In one embodiment 50-2000 μl of PBS are added to the sample tube, in a preferred embodiment 250-1000 μl of PBS are added, and most preferably 250 μl of PBS are added. The sample is incubated at room temperature to allow extraction of sample from the swab. In one embodiment the sample is incubated from 1 min to 24 hrs, in a preferred embodiment the sample is incubated from 10 min to 2 hr, in a most preferred embodiment the sample is incubated for 1 hour. Sample incubation varies dependent upon saliva concentration, sample surface, and age of sample. Due to the low cost of hAMY1A ICS tests, samples can be repeatedly tested to determine saliva extraction from the sample. In one embodiment samples are assayed every 10 min, in a preferred embodiment samples are assayed at 10 min, 30 min, 3 hrs, and 24 hrs, in a most preferred embodiment samples are assayed at 1 hr and 24 hrs. ICS tests are developed for approximately 10 minutes and scored for the presence of hAMY1A as previously described. To quantitatively assess hAMY1A concentration, dilutions may be made and compared to positive control samples of a known hAMY1A concentration.

We have described elongated dipstick tests herein for purposes of illustration only. However, other designs are possible including annular ring designs, blot cards, and test sticks (immersed in sample/conjugate antibody solution). Fluid migration may be monitored by control line intensity, dye front, or color changes when damp. Kits described herein are for demonstration purposes and additional components may be added.

### REFERENCES

All references are listed herein for the convenience of the reader. Each is incorporated by reference in its entirety.
1. Quarino, et al., “An ELISA method for the identification of salivary amylase.” J. Forensic. Sci. 50:873-6 (2005).
2. Aluoch, et al., “Development of an oral biosensor for salivary amylase using a monodispersed silver for signal amplification.” Anal Biochem. 340:136-44 (2005).
3. Muller, [Amylase excretion in the feces] Z. Gastroenterol. 18:198-202 (1980).
4. Heitlinger, et al., “Mammary amylase: a possible alternate pathway of carbohydrate digestion in infancy,” Pediatric Research. 17:15-8 (1983).

What is claimed is:

1. A device for detecting human saliva, comprising:
a) a wettable material,
b) a sample comprising a first human salivary amylase (hAMY1A) antibody, where said first antibody is labeled for detection, and
c) a second hAMYlA antibody immobilized to said wettable material at a single location,
wherein the first antibody and the second antibody can simultaneously bind to human salivary amylase, but do not bind pancreatic amylase or non-human salivary amylase.

2. The device of claim 1, further comprising an immobilized control antibody adjacent said second hAMY1A antibody wherein said control antibody binds detection antibody in the presence or absence of hAMY1A.

3. The device of claim 1, wherein said wettable material is a lateral flow immunochromatographic strip (ICS) test comprising:
a) a first pad comprising the first hAMY1A antibody labeled for detection;
) a membrane comprising the second hAMY1A antibody immobilized in a test line, and
c) an second pad, wherein said membrane (b) connects said first pad (a) to said second pad.

4. The ICS test of claim 3, further comprising a control antibody immobilized in a control line adjacent said test line on membrane (b) wherein said control antibody binds detection antibody in the presence or absence of hAMY1A.

5. The ICS test of claim 3, wherein said first antibody and said second antibody are combinations selected from the group consisting of polyclonal antibodies, monoclonal antibodies, recombinant antibodies, AMY1, AMY2, and AMY16C.

6. The ICS test of claim 3, wherein said first antibody is labeled with colloidal gold, streptavidin, biotin, microspheres, latex beads, peroxidase, horse radish peroxidase (HRP), streptavidin-labeled HRP, phosphatase, alkaline phosphatase (AP), chromogenic labels, fluorescent labels, phosphorescent labels, or chemiluminescent labels.

7. The ICS test of claim 3, wherein said first antibody and second antibody are selected from the group consisting of AMY1 immobilization antibody and AMY2 detection antibody, AMY1 immobilization antibody and AMY16C detection antibody, AMY2 immobilization antibody and AMY1 detection antibody, AMY2 immobilization antibody and AMY16C detection antibody, AMY16C immobilization antibody and AMY1 detection antibody, AMY16C immobilization antibody and AMY2 detection antibody.

8. The ICS test of claim 3, wherein one or more ICS test is assembled in a cassette.

9. The ICS test of claim 3, wherein one or more ICS test is contained in a kit optionally comprising sample swabs, swab wetting solution, sample tubes, sample tube holder, scissors, sample extraction buffer, sample running buffer, transfer pipet, completed ICS test documentation envelope, syringes, immunochromatographic solutions, additional detection antibodies, secondary enzyme assay reagents, and instructions.

10. A method for detecting the presence of human saliva comprising:
a) swabbing a surface suspected of containing saliva with a swab;
b) resuspending the contents of the swab (a) in buffer;
c) applying buffer (b) to a device for detecting human saliva comprising:
i) a first human salivary amylase (hAMY1A) antibody labeled for detection, and
ii) a second hAMY1A antibody immobilized to a wettable material at a single separate location,
wherein the first antibody and the second antibody can simultaneously bind to human salivary amylase, but do not bind pancreatic amylase or non-human salivary amylase; and
d) detecting the presence or absence of concentrated detection antibodies at the second immobilized antibody wherein the presence of a test line confirms the presence of human saliva.

11. The method of claim 10, wherein said first antibody and second antibody are combinations selected from the group consisting of polyclonal antibodies, monoclonal antibodies, recombinant antibodies, AMY1, AMY2, and AMY16C.

12. The method of claim 10, wherein said first antibody is labeled with colloidal gold, streptavidin, biotin, microspheres, latex beads, peroxidase, streptavidin-labeled horse radish peroxidase (HRP), phosphatase, alkaline phosphatase (AP), chromogenic labels, fluorescent labels, phosphorescent labels, or chemiluminescent labels.

13. The method of claim 10, wherein said first antibody and second antibody are selected from the group consisting of AMY1 immobilization antibody and AMY2 detection antibody, AMY1 immobilization antibody and AMY16C detection antibody, AMY2 immobilization antibody and AMY1 detection antibody, AMY2 immobilization antibody and AMY16C detection antibody, AMY16C immobilization antibody and AMY1 detection antibody, AMY16C immobilization antibody and AMY2 detection antibody.

14. The method of claim 10, wherein said device (c) is a immunochromatographic strip (ICS) test comprising:
i) a first pad comprising the first detection antibody that binds hAMYlA;
ii) a wettable material comprising the second immobilized hAMYlA antibody in a test line, and
iii) a second pad, wherein said wettable material (ii) connects said first pad (i) to said absorbent pad.

15. The method of claim 13, further comprising a comparison of the sample ICS test to one or more ICS tests comprising known concentrations of hAMY1A.

16. The method of claim 13, wherein one or more test strips comprise 0 ng/ml to 100 ng/ml of hAMY1A or concentrations there between.

17. A method of manufacturing one or more human salivary immunochromatographic strip (ICS) tests comprising:
a) placing a backing on one surface;
b) adhering to the backing:
i) a first pad comprising a first human salivary amylase (hAMY1A) antibody labeled for detection,
ii) a second pad opposite the first pad, and
iii) a membrane comprising a second hAMY1A antibody immobilized in a test line wherein said chromatographic membrane contacts both first and second pad, wherein the first antibody and the second antibody can simultaneously bind to human salivary amylase, but do not bind pancreatic amylase or non-human salivary amylase;
c) laminating said components to said backing creating an assembled card; and
d) cutting said assembled card into longitudinal strips to create one or more ICS test strips.

18. The method of claim 17, wherein said first pad is placed atop or adjacent to a third sample pad.

19. The method of claim 17, wherein said first antibody and second antibody are combinations selected from the group consisting of polyclonal antibodies, monoclonal antibodies, recombinant antibodies, AMY1, AMY2, and AMY16C.

20. The method of claim 17, wherein said first antibody is labeled with colloidal gold, streptavidin, biotin, microspheres, latex beads, peroxidase, horse radish peroxidase (HRP), streptavidin-labeled HRP, phosphatase, alkaline phosphatase (AP), chromogenic labels, fluorescent labels, phosphorescent labels, or chemiluminescent labels.

21. The method of claim 17, wherein said first antibody and second antibody are selected from the group consisting of AMY1immobilization antibody and AMY2 detection antibody, AMY1 immobilization antibody and AMY16C detection antibody, AMY2 immobilization antibody and AMY1 detection antibody, AMY2 immobilization antibody and AMY16C detection antibody, AMY16C immobilization antibody and AMY1 detection antibody, AMY16C immobilization antibody and AMY2 detection antibody.

22. The method of claim 17, wherein one or more ICS test is assembled in a cassette.

23. The method of claim 17, wherein one or more ICS test is provided in a kit optionally comprising sample swabs, swab wetting solution, sample tubes, sample tube holder, scissors, sample extraction buffer, sample running buffer, transfer pipet, completed ICS test documentation envelope, syringes, immunochromatographic solutions, additional detection antibodies, secondary enzyme assay reagents, and instructions.
